(19) United States
(12) Patent Application Publication
(10) Pub. No.: US 2020/0400697 A1
(43) Pub. Date: Dec. 24, 2020

(54) METHOD OF OPERATING AN ANALYTICAL LABORATORY

(71) Applicant: Roche Diagnostics Operations, Inc., Indianapolis, IN (US)

(72) Inventors: Achim Sinz, Horb (DE); Marco Maetzler, Belmont, CA (US)

(73) Assignee: Roche Diagnostics Operations, Inc., Indianapolis, IN (US)

(21) Appl. No.: 16/903,537

(22) Filed: Jun. 17, 2020

(30) Foreign Application Priority Data
Jun. 24, 2019 (EP) 19181942.4

(51) Int. Cl. G01N 35/00; G01N 33/483; G16H 10/40

(52) U.S. Cl. CPC G01N 35/0095; G01N 35/00613; G01N 33/483; G01N 2035/0401; G01N 2035/00227; G01N 2035/00841; G16H 10/40

(57) ABSTRACT

A method of operating an analytical laboratory is presented. The method comprises recording time at which samples are first identified; retrieving an order list comprising test orders for the samples; retrieving a degradation limit to each test order; determining workflows for each sample; instructing the laboratory instruments to carry out the test orders according to the workflows. Determining the workflows comprises i) determining target instruments capable of carrying out the test orders; ii) determining a sequence/timing of the test orders; iii) calculating an estimated completion time for each test order; iv) determining a lead time for each sample and test order; v) prioritizing test orders if the lead time exceeds the degradation limit. Steps ii) to v) are repeated until the lead time doesn't exceed the degradation limit for any of the test orders; or until steps ii) to v) have been repeated for a number N of iterations.

### CROSS-REFERENCE TO RELATED APPLICATIONS

This application claims priority to EP 19181942.4, filed Jun. 24, 2019, which is hereby incorporated by reference.

### BACKGROUND

The present disclosure generally relates to method of operating an analytical laboratory and, in particular, to an in-vitro diagnostic laboratory.

In vitro diagnostic testing has a major effect on clinical decisions, providing physicians with pivotal information. In analytical laboratories, in particular, in-vitro diagnostic laboratories, a multitude of analyses on biological samples are executed by laboratory instruments in order to determine physiological and biochemical states of patients, which can be indicative of a disease, nutrition habits, drug effectiveness, organ function and the like.

According to established laboratory procedures in complex analytical laboratories, a plurality of instruments process biological samples according to test orders, each test order defining one or more processing steps to be carried out on the biological sample. After the biological sample has been received and identified by a pre-analytical laboratory instrument, a control unit retrieves the corresponding test orders and determines which instruments are required to process the biological sample according to the test order(s). Having identified the required instrument(s), the control unit determines a sample workflow for each sample according to the test order(s). The sample workflow comprising a sequence and/or timing of carrying out the one or more test orders by the one or more analytical instruments.

In current laboratories, biological samples are processed, transported and sometimes even stored at room temperature. Once all test orders related to the biological sample are completed, the sample is stored in an archive (usually refrigerated) or discarded.

However, it has been observed that certain analytes and biological samples degrade over time, in particular if stored at room temperature. Therefore, the validity of certain analytical tests can no longer be guaranteed after the sample has been stored beyond a certain period of time, hereafter referred to as degradation limit.

Known solutions exist which track the temperature of an entire analytical laboratory and/or an analytical instrument, raising an alarm or flagging test results if a critical temperature is exceeded. The disadvantage of such a solution is that it can only address a general problem applicable to all samples in the laboratory/analytical instrument. However, sample degradation does not only occur due to exceeding a critical temperature but also at normal operating temperature of an analytical laboratory.

One prior art system identifies invalid analysis results. This system is reactive to biological samples with exceeded degradation limits. With such a reactive system, a substitute biological sample needs to be provided for each sample with an exceeded degradation limit. However, providing a substitute biological sample often leads to delays in the total turn around time (the time by which the analysis result becomes available) and/or inconvenience to the patient since biological sample needs to be collected again. Furthermore, in case of newborn babies or in forensics, providing a substitute biological sample may not even possible.

Therefore, there is a need for a method of operating an analytical laboratory such as, an analytical laboratory system, which can proactively prevent the degradation limit of biological sample(s) to be exceeded.

### SUMMARY

According to the present disclosure, a method of operating an analytical laboratory. The method can comprise receiving and identifying a plurality of biological samples; upon identifying the biological samples, recording data indicative of a time at which the biological samples were first identified; retrieving by a control unit of the analytical laboratory an order list from a data storage unit comprising one or more test orders corresponding to each biological sample; retrieving from the data storage unit by the control unit data indicative of a degradation limit corresponding to each test order of the order list; determining by the control unit sample workflows corresponding to each biological sample and the order list; and instructing by the control unit one or more laboratory instruments of the analytical laboratory to carry out the test orders according to the sample workflows. The determination of the sample workflows comprises i) determining a target list of one or more laboratory instruments of the analytical laboratory for carrying out the one or more test orders, ii) determining a sequence and/or timing of carrying out the one or more test orders by the target list of laboratory instruments, iii) calculating an estimated completion time for each test order based on the sequence and/or timing of carrying out the one or more test orders, iv) determining a lead time corresponding to each biological sample for each test order, the lead time being the time period between the time at which the biological sample was first identified and the estimated completion time of the respective test order, and v) prioritizing one or more test orders from the order list if the lead time exceeds the degradation limit corresponding to the respective test order, repeating steps ii) to v) until the lead time doesn't exceed the degradation limit for any of the test orders or until steps ii) to v) have been repeated for a number N of iterations.

Accordingly, it is a feature of the embodiments of the present disclosure to provide a method of operating an analytical laboratory such as, an analytical laboratory system, which can proactively prevent the degradation limit of biological sample(s) to be exceeded. Other features of the embodiments of the present disclosure will be apparent in light of the description of the disclosure embodied herein.

### BRIEF DESCRIPTION OF THE SEVERAL VIEWS OF THE DRAWINGS

The following detailed description of specific embodiments of the present disclosure can be best understood when read in conjunction with the following drawings, where like structure is indicated with like reference numerals and in which:

FIG. 1 illustrates a highly schematic block diagram of an analytical laboratory according to an embodiment of the present disclosure.

FIG. 2 illustrates a flowchart illustrating the disclosed method according to an embodiment of the present disclosure.

FIG. 3 illustrates a flowchart illustrating the disclosed method according to another embodiment of the present disclosure.

FIG. 4 illustrates a flowchart illustrating the disclosed method according to a further embodiment of the present disclosure.

FIG. 5 illustrates a flowchart illustrating the disclosed method according to still yet another embodiment of the present disclosure.

FIG. 6 illustrates a highly schematic block diagram of a pre-analytical laboratory instrument of the disclosed analytical laboratory according to an embodiment of the present disclosure.

FIG. 7 illustrates a highly schematic block diagram of a pre-analytical laboratory instrument of the disclosed analytical laboratory according to another embodiment of the present disclosure.

FIG. 8 illustrates a highly schematic block diagram of an analytical laboratory instrument of the disclosed analytical laboratory according to an embodiment of the present disclosure.

FIG. 9 illustrates a highly schematic block diagram of a post-analytical laboratory instrument of the disclosed analytical laboratory according to an embodiment of the present disclosure.

### DETAILED DESCRIPTION

In the following detailed description of the embodiments, reference is made to the accompanying drawings that form a part hereof, and in which are shown by way of illustration, and not by way of limitation, specific embodiments in which the disclosure may be practiced. It is to be understood that other embodiments may be utilized and that logical, mechanical and electrical changes may be made without departing from the spirit and scope of the present disclosure.

The use of the ‘a’ or ‘an’ are employed to describe elements and components of the embodiments herein. This is done merely for convenience and to give a general sense of the inventive concepts. This description should be read to include one or at least one and the singular also includes the plural unless it is obvious that it is meant otherwise.

As used herein qualifiers such as ‘about,’ ‘approximately,’ and ‘substantially’ are intended to signify that the item or value being qualified is not limited to the exact value or amount specified, but includes some slight variations or deviations therefrom, caused by measuring error or imprecision, manufacturing tolerances, stress exerted on various parts, wear and tear, and combinations thereof, for example.

The terms ‘sample’, ‘patient sample’ and ‘biological sample’ can refer to material(s) that may potentially contain an analyte of interest. The patient sample can be derived from any biological source, such as a physiological fluid, including blood, saliva, ocular lens fluid, cerebrospinal fluid, sweat, urine, stool, semen, milk, ascites fluid, mucous, synovial fluid, peritoneal fluid, amniotic fluid, tissue, cultured cells, or the like. The patient sample can be pretreated prior to use, such as preparing plasma from blood, diluting viscous fluids, lysis or the like. Methods of treatment can involve filtration, distillation, concentration, inactivation of interfering components, and the addition of reagents. A patient sample may be used directly as obtained from the source or used following a pretreatment to modify the character of the sample. In some embodiments, an initially solid or semi-solid biological material can be rendered liquid by dissolving or suspending it with a suitable liquid medium. In some embodiments, the sample can be suspected to contain a certain antigen or nucleic acid.

The term ‘analyte’ can be a component of a sample to be analyzed, e.g., molecules of various sizes, ions, proteins, metabolites, nucleic acid sequences and the like. Information gathered on an analyte may be used to evaluate the impact of the administration of drugs on the organism or on particular tissues or to make a diagnosis. Thus, ‘analyte’ can be a general term for substances for which information about presence, absence, and/or concentration is intended. Examples of analytes are e.g., glucose, coagulation parameters, endogenic proteins (e.g., proteins released from the heart muscle), metabolites, nucleic acids and so on.

The term ‘laboratory instrument’ as used herein can encompass any apparatus or apparatus component operable to execute and/or cause the execution of one or more processing steps/workflow steps on one or more biological samples and/or one or more reagents. The expression ‘processing steps’ thereby can refer to physically executed processing steps such as centrifugation, aliquotation, sample analysis and the like. The term ‘instrument’ can cover pre-analytical instruments, post-analytical instruments, analytical instruments and laboratory middleware.

The term ‘post-analytical instrument’ as used herein can encompass any apparatus or apparatus component that can be configured to perform one or more post-analytical processing steps/workflow steps comprising—but not limited to—sample unloading, transport, recapping, decapping, temporary storage/buffering, archiving (refrigerated or not), retrieval and/or disposal.

The term ‘pre-analytical instrument’ as used herein can encompass any apparatus or apparatus component that can be configured to perform one or more pre-analytical processing steps/workflow steps comprising—but not limited to—centrifugation, resuspension (e.g., by mixing or vortexing), capping, decapping, recapping, sorting, tube type identification, sample quality determination and/or aliquotation steps. The processing steps may also comprise adding chemicals or buffers to a sample, concentrating a sample, incubating a sample, and the like.

The term ‘analyzer’/‘analytical instrument’ as used herein can encompass any apparatus or apparatus component configured to obtain a measurement value. An analyzer can be operable to determine via various chemical, biological, physical, optical or other technical procedures a parameter value of the sample or a component thereof. An analyzer may be operable to measure the parameter of the sample or of at least one analyte and return the obtained measurement value. The list of possible analysis results returned by the analyzer comprises, without limitation, concentrations of the analyte in the sample, a digital (yes or no) result indicating the existence of the analyte in the sample (corresponding to a concentration above the detection level), optical parameters, DNA or RNA sequences, data obtained from mass spectrometry of proteins or metabolites and physical or chemical parameters of various types. An analytical instrument may comprise units assisting with the pipetting, dosing, and mixing of samples and/or reagents. The analyzer may comprise a reagent-holding unit for holding reagents to perform the assays. Reagents may be arranged for example in the form of containers or cassettes containing individual reagents or group of reagents, placed in appropriate receptacles or positions within a storage compartment or conveyor. It may comprise a consumable feeding unit. The analyzer may comprise a process and detection system whose workflow is optimized for certain types of analysis. Examples of such analyzer can be clinical chemistry analyzers, coagulation chemistry analyzers, immunochemistry analyzers, urine analyzers, nucleic acid analyzers, used to detect the result of chemical or biological reactions or to monitor the progress of chemical or biological reactions.

The term ‘laboratory middleware’ as used herein can refer to any physical or virtual processing device configurable to control a laboratory instrument or system comprising one or more laboratory instruments in a way that workflow(s) and workflow step(s) are conducted by the laboratory instrument/system. The laboratory middleware may, for example, instruct the laboratory instrument/system to conduct pre-analytical, post analytical and analytical workflow(s)/workflow step(s). The laboratory middleware may receive information from a data management unit regarding which steps need to be performed with a certain sample. In some embodiments, the laboratory middleware can be integral with a data management unit, can be comprised by a server computer and/or be part of one laboratory instrument or even distributed across multiple instruments of the analytical laboratory. The laboratory middleware may, for instance, be embodied as a programmable logic controller running a computer-readable program provided with instructions to perform operations.

The term ‘sample transportation system’ as used herein can encompass any apparatus or apparatus component that can be configured to transport sample carriers (each holding one or more sample containers) between laboratory instruments. In particular, the sample transportation system can be a one dimensional conveyor-belt based system, a two-dimensional transportation system (such as a magnetic sample carrier transport system) or a combination thereof.

An ‘analytical laboratory’ as used herein can comprise a system comprising one or more analytical; pre- and post-analytical laboratory instruments; a sample transportation system and/or a laboratory middleware.

The term ‘analysis or ‘analytical test’ as used herein can encompass a laboratory procedure characterizing a parameter of a biological sample for qualitatively assessing or quantitatively measuring the presence or amount or the functional activity of an analyte.

The ‘term consumable’ can comprise—but is not limited—to reagents, system fluids, quality control material, calibrator materials, microplates/microwell plates, reaction vessels, measurement cuvettes, sample tubes, pipetting tips, and the like.

As used herein, the term “calibrator” can refer to a composition containing a known concentration of an analyte for use in determining the concentration of the analyte in a sample containing an unknown concentration of the analyte.

The term ‘communication network’ as used herein can encompass any type of wireless network, such as a WiFi™, GSM™, UMTS or other wireless digital network or a cable based network, such as Ethernet™ or the like. In particular, the communication network can implement the Internet protocol (IP). For example, the communication network can comprise a combination of cable-based and wireless networks.

The term ‘remote system’ or ‘server’ as used herein can encompass any physical machine or virtual machine having a physical or virtual processor, capable of receiving, processing and sending data. A server can run on any computer including dedicated computers, which individually are also often referred to as ‘the server’ or shared resources such as virtual servers. In many cases, a computer can provide several services and have several servers running. Therefore, the term server can encompass any computerized device that shares a resource with one or more client processes. Furthermore, the terms ‘remote system’ or ‘server’ can encompass a data transmission and processing system distributed over a data network (such as a cloud environment).

The term ‘user interface’ as used herein can encompass any suitable piece of software and/or hardware for interactions between an operator and a machine, including but not limited to a graphical user interface for receiving as input a command from an operator and also to provide feedback and convey information thereto. Also, a system/device may expose several user interfaces to serve different kinds of users/operators.

The term ‘Quality control’ or ‘analytical quality control’ can refer to all those processes and procedures designed to ensure that the results of laboratory analysis (analytical tests) are consistent, comparable, accurate and within specified limits of precision.

Embodiments disclosed herein address the need for a method of operating an analytical laboratory, respectively an analytical laboratory system which can proactively prevent the degradation limit of biological sample(s) from being exceeded, by determining/re-determining the sample workflows for test orders in an analytical laboratory taking into account a projected lead time of the biological samples, prioritizing test orders of biological samples which otherwise would exceed the degradation limit.

The disclosed method of operating an analytical laboratory is presented. The method can comprises the steps of receiving and identifying a plurality of biological samples and upon identifying the biological samples, recording data (e.g., a timestamp) indicative of a time at which the biological samples were first identified. According to embodiments disclosed, the first identification and, hence, the timestamp recorded of the biological samples can be performed at collection of the biological samples (e.g., by the phlebotomist) or by a laboratory instrument such as, for example, a pre-analytical laboratory instrument.

The method can also comprise retrieving by a control unit of the analytical laboratory an order list from a data storage unit comprising one or more test orders corresponding to each biological sample. A test order can define one or more processing steps to determine presence, absence, and/or concentration of an analyte in the biological sample.

The method can also comprise retrieving from the data storage unit by the control unit data indicative of a degradation limit corresponding to each test order of the order list. The degradation limit can be indicative of a maximum time a biological sample may be stored after which validity of the test order can no longer be guaranteed. According to embodiments disclosed herein, the degradation limit can comprise multiple time limits, each associated with a particular storage condition (e.g., temperature/humidity ranges, capped/uncapped sample containers).

The method can also comprise determining by the control unit sample workflows corresponding to each biological sample and the order list. The sample workflow can comprise a sequence and/or timing of carrying out the one or more test orders by analytical instruments of the analytical laboratory.

The method can also comprise instructing by the control unit one or more analytical instruments of the analytical laboratory to carry out the test orders according to the sample workflow.

According to the disclosed method, the determination of the sample workflows can comprise the following: i) determining a target list of one or more analytical instruments of the analytical laboratory capable of carrying out the one or more test orders. According to embodiments disclosed herein, the target list of analytical instruments can be determined based on the availability and capability of the analytical instruments to process the biological samples according to the corresponding test orders. ii) Determine a sequence and/or timing of carrying out the one or more test orders by the target list of analytical instruments. According to embodiments disclosed herein, sequence and/or timing of carrying out the one or more test orders can be determined by the control unit using a set of rules taking in consideration aspects comprising (but not limited to) decontamination level of each instrument; load balancing between instruments; and/or urgency of test orders. iii) Calculating an estimated completion time for each test order based on the sequence and/or timing of carrying out the one or more test orders. According to embodiments disclosed herein, the completion time can be calculated based on estimated duration of each processing step required to complete the respective test order, the duration being estimated based on manufacturer specifications; statistical values and/or estimated based on current processing capacity/speed of the respective laboratory instruments. According to various embodiments disclosed herein, the estimated completion time can further comprise a transportation time to the respective laboratory instruments and/or a reserve time accounting for unforeseen variations of the actual processing time of the biological samples as compared to the estimated duration of each processing step. iv) Determining a lead time corresponding to each biological sample for each test order. The lead time can be the time period between the time at which the biological sample was first identified and the estimated completion time of the respective test order. v) Prioritizing one or more test orders from the order list if the lead time exceeds the degradation limit corresponding to the respective test order. According to embodiments disclosed herein, the step of prioritizing one or more test orders from the order list can comprise adjusting the sequence and/or timing of carrying out the one or more test order such that the test order with a lead time exceeding the degradation limit can be carried out at an earlier time as compared to the sample workflow before the prioritization. The steps ii) to v) can be repeated until the lead time doesn't exceed the degradation limit for any of the test orders or until the steps ii) to v) have been repeated for a number N of iterations.

The method and the system disclosed herein can be advantageous as they can proactively estimate the lead time of each biological sample and in N iterations prioritize the processing of biological samples which would exceed their degradation limit. In such a way, the waste of biological samples and/or the inconvenience caused to patient(s) can be reduced and/or sample result turnaround time(s) can be greatly improved.

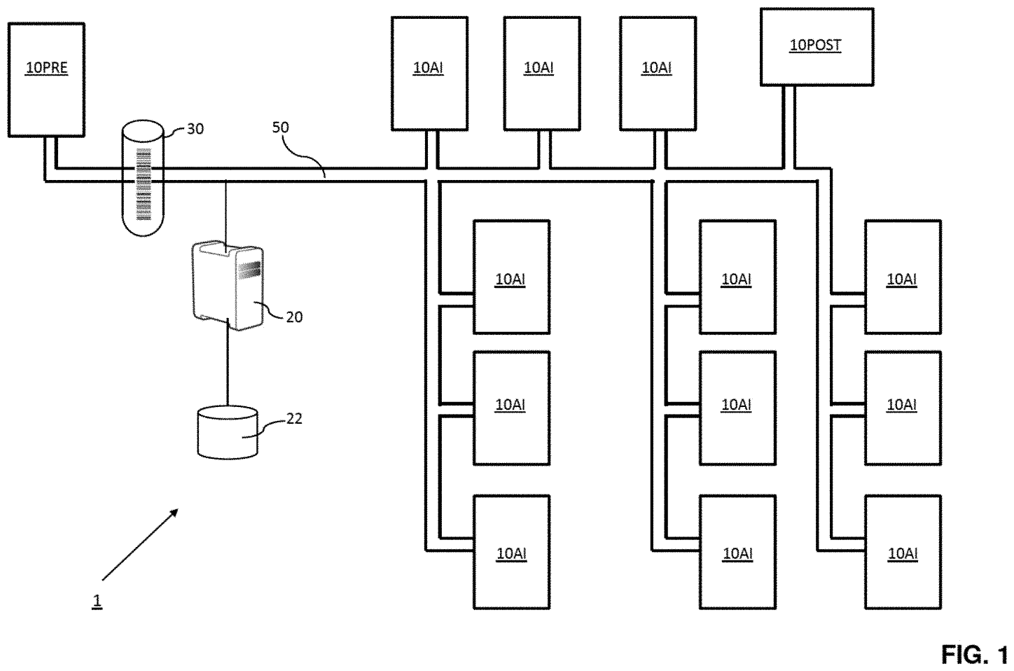

Referring initially to FIG. 1, FIG. 1 shows a highly schematic block diagram of an embodiment of the disclosed analytical laboratory 1. As shown on the block diagram of FIG. 1, embodiments of the disclosed analytical laboratory 1 for processing biological sample(s) can comprise a plurality of laboratory instruments 10AI, 10PRE, 10POST and a control unit 20 communicatively connected by a communication network. The plurality of laboratory instruments 10AI, 10PRE, 10POST can be configured to execute processing steps on the biological samples according to instructions from the control unit 20.

The pre-analytical instruments 10PRE comprised by the analytical laboratory 1 may be one or more from the list comprising: an instrument for centrifugation of samples, a capping-, decapping- or recapping instrument, aliquoter, a buffer to temporarily store biological samples or aliquots thereof.

The post-analytical instruments 10POST comprised by the analytical laboratory 1 may be one or more from the list comprising: a recapper, an unloader for unloading a sample from an analytical system and/or transporting the sample to a data storage unit or to a unit for collecting biological waste.

According to various embodiments of the disclosed analytical laboratory 1, the plurality of laboratory instruments 10AI, 10PRE, 10POST may be identical or different instruments such as clinical- & immunochemistry analyzers, coagulation chemistry analyzers, immunochemistry analyzers, urine analyzers, nucleic acid analyzers, hematology instruments and the like.

The control unit 20 can be configured to control the analytical laboratory 1 to carry out the steps of one or more of the methods herein disclosed and can be communicatively connected to the data storage unit 22.

As shown on FIG. 1, the analytical laboratory 1 can further comprise a sample transportation system 50 interconnecting the plurality of laboratory instruments 10AI, 10PRE, 10POST. According to embodiments disclosed herein, the sample transportation system 50 can be a one-dimensional conveyor-belt based system. According to further embodiments disclosed (but not illustrated), the sample transportation system 50 can be a two-dimensional transportation system (such as a magnetic sample carrier transport system). The analytical laboratory 1 can be configured to carry out the method according to the embodiments disclosed herein.

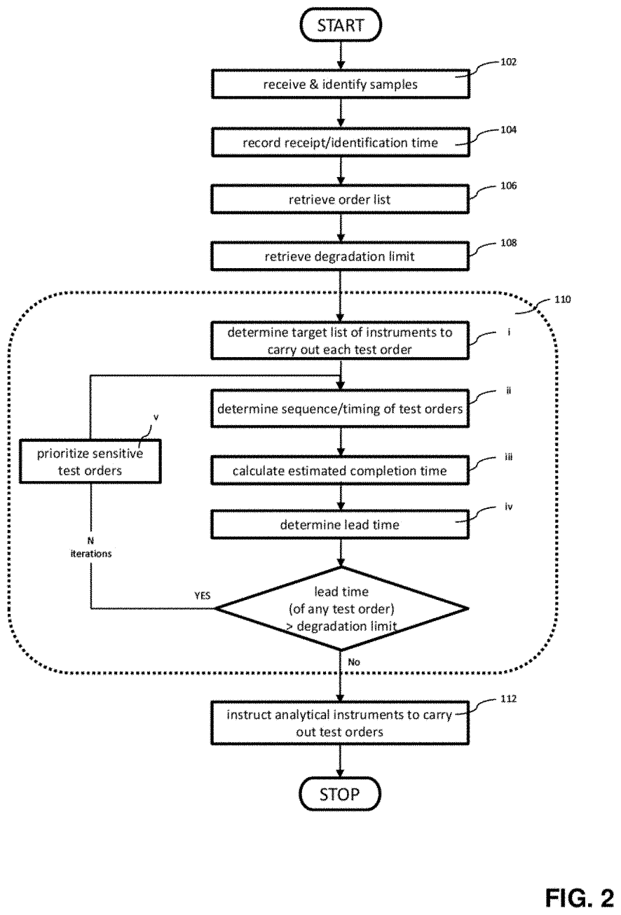

Turing now to FIGS. 2-5, embodiments of the disclosed method of operating an analytical laboratory will be described with reference to the figures.

As shown on FIG. 2, in a first step 102 of the claimed method, samples can be received and identified. According to embodiments disclosed herein, the receipt and identification of samples can be performed by a phlebotomist collecting the biological sample(s), a laboratory technician handling the biological sample(s) and/or the first laboratory instrument 10AI, 10PRE, 10POST of the analytical laboratory 1 that the biological sample(s) can be loaded onto such as, for example, a pre-analytical laboratory instrument 10PRE. Thereafter, in a step 104, upon identifying the biological samples, data can be recorded indicative of a time at which the biological samples were first identified. Analogous to the embodiments mentioned above, the data indicative of a time at which the biological samples were first identified can be recorded by the phlebotomist collecting the biological sample(s), a laboratory technician handling the biological sample(s), the control unit 20 being configured for receiving input indicative of a time at which the biological samples were collected, for example via a mobile app, a graphical user interface (GUI), or a speech recognition system directly or indirectly communicatively connected to the control unit 20. Alternatively, or additionally, the data indicative of a time at which the biological samples were first identified can be recorded by the one or more laboratory instrument(s) 10PRE, 10AI, 10POST configured to receive and identify biological sample(s).

In order to determine which processing steps need to be carried out on each biological sample, in a step 106, the control unit 20 can retrieve an order list from a data storage unit 22 comprising one or more test orders corresponding to each biological sample. A test order can define one or more processing steps to determine presence, absence, and/or concentration of an analyte in the biological sample.

To determine the sensitivity of a test order to sample degradation (due to extended storage), in a step 108, data indicative of a degradation limit corresponding to each test order of the order list can be retrieved by the control unit 20 from the data storage unit 22. The degradation limit can be indicative of a maximum time a biological sample may be stored after which validity of the test order may no longer be guaranteed. According to embodiments disclosed herein, the degradation limit can comprise multiple time limits, each associated with a particular storage condition (e.g., temperature/humidity ranges and/or capped/uncapped sample containers).

Thereafter, in a step 110, the control unit 20 can determine sample workflows for each biological sample. A sample workflow can comprise a sequence and/or timing of carrying out the one or more test orders by analytical instruments of the analytical laboratory 1. Furthermore, according to embodiments disclosed herein, the sample workflow can further comprise instructions which, when executed by the respective laboratory instruments, can cause the laboratory instruments 10AI, 10PRE, 10POST to carry out the processing steps as defined by the test orders.

As shown on FIG. 1 as well, the step 110 of determining sample workflows for each biological sample can comprise several substeps. In a substep 110 i), the control unit 20 can determine a target list of one or more analytical instruments 10AI of the analytical laboratory 1 for carrying out the one or more test orders. According to embodiments disclosed herein, the target list of analytical instruments 10AI can be determined based on the availability and capability thereof to process the biological samples according to the corresponding test orders. According to particular embodiments disclosed herein, determining a target list of one or more analytical instruments 10AI of the analytical laboratory 1 capable of carrying out the one or more test orders can comprise the steps of: determining whether the laboratory instrument 10AI, 10PRE, 10POST is powered on and not in a low-power mode; determining whether all modules of the laboratory instrument 10AI, 10PRE, 10POST required to carry out the respective test order are operational; determining whether all consumables required to carry out the respective test order are available; and/or determining whether all quality control and/or calibration values of the laboratory instrument 10AI, 10PRE, 10POST are up-to-date and valid.

In a substep 110 ii), the control unit 20 can determine a sequence and/or timing of carrying out the one or more test orders by the one or more analytical instruments 10AI of the target list. According to embodiments disclosed herein, the sequence and/or timing of carrying out the one or more test orders can be determined by the control unit 20 using a set of rules (also called a rule engine) taking in consideration aspects comprising (but not limited to) decontamination level of each instrument 10AI (a highly decontaminated instrument may receive biological samples before any instrument of lower decontamination level); load balancing between instruments; and/or urgency of test orders and/or a multitude of laboratory specific configuration rules.

In substep 110 iii), the control unit 20 can calculate an estimated completion time for each test order based on the sequence and/or timing of carrying out the one or more test orders. According to embodiments disclosed herein, the estimated completion time for each test order can be determined based on estimated duration of each processing step of each test order preceding the current test order for the respective laboratory instruments 10AI, 10PRE, 10POST and the estimated duration of each processing step of the current test order. In other words, the estimated completion time can take into consideration all processing steps that need to be carried out before the current test order and the processing steps of the current test order. This estimation can be based on historical data and/or instrument specification(s) and/or a setting by a laboratory technician and/or estimated based on current processing capacity/speed of the respective laboratory instruments. According to embodiments disclosed herein, the estimated duration of each processing step can be (re)adjusted based on measured duration of the processing step(s).

According to further embodiments disclosed herein, the estimated completion time can further comprise a transportation time to the respective laboratory instruments 10AI, 10PRE, 10POST and/or a reserve time accounting for unforeseen variations of the actual processing time of the biological samples as compared to the estimated duration of each processing step.

Having determined the estimated completion time, in a subsequent substep 110 iv), the control unit 20 can determine a lead time corresponding to each biological sample for each test order. The lead time as used herein can refer to the time period between the time at which the biological sample was first identified and the estimated completion time of the respective test order.

If the lead time exceeds the degradation limit corresponding to a test order, in substep 110 v), the control unit 20 can prioritize the respective test orders from the order list. In other words, if the control unit 20 estimates that by the time the biological sample can be processed according to the test order, the sample would be degraded, the particular test order can be prioritized. According to embodiments disclosed herein, the step 110 v) of prioritizing one or more test orders from the order list can comprises adjusting the sequence and/or timing of carrying out the one or more test orders such that the test order with a lead time exceeding the degradation limit can be carried out at an earlier time as compared to the sample workflow before the prioritization. It can be noted that depending on the particular test orders of the analytical laboratory 1, the prioritization may or may not affect test orders of other biological samples.

In order to ensure that the prioritization reached its objective and did not negatively affect other test orders, steps 110 ii) to v) can be repeated until the lead time doesn't exceed the degradation limit for any of the test orders. Since there may be occasions when, despite (re) prioritization of test orders, a set of sample workflows cannot be determined where the lead time doesn't exceed the degradation limit for any of the test orders, steps 110 ii) to v) can be repeated for a number N of iterations.

Once the lead time doesn't exceed the degradation limit for any of the test orders or once steps ii) to v) have been repeated for a number N of iterations, in a step 112, the control unit 20 can instruct the one or more analytical instruments 10AI of the analytical laboratory to carry out the test orders according to the sample workflows.

According to embodiments disclosed herein, the control unit 20 can associate a processing priority level with each test order and—within the step of prioritizing one or more test orders from the order list—can increase the processing priority level for each test order with a lead time exceeding the degradation limit. Correspondingly, the step 112 can comprise instructing by the control unit 20 one or more analytical instruments 10AI of the analytical laboratory to carry out the test orders according to the respective processing priority level.

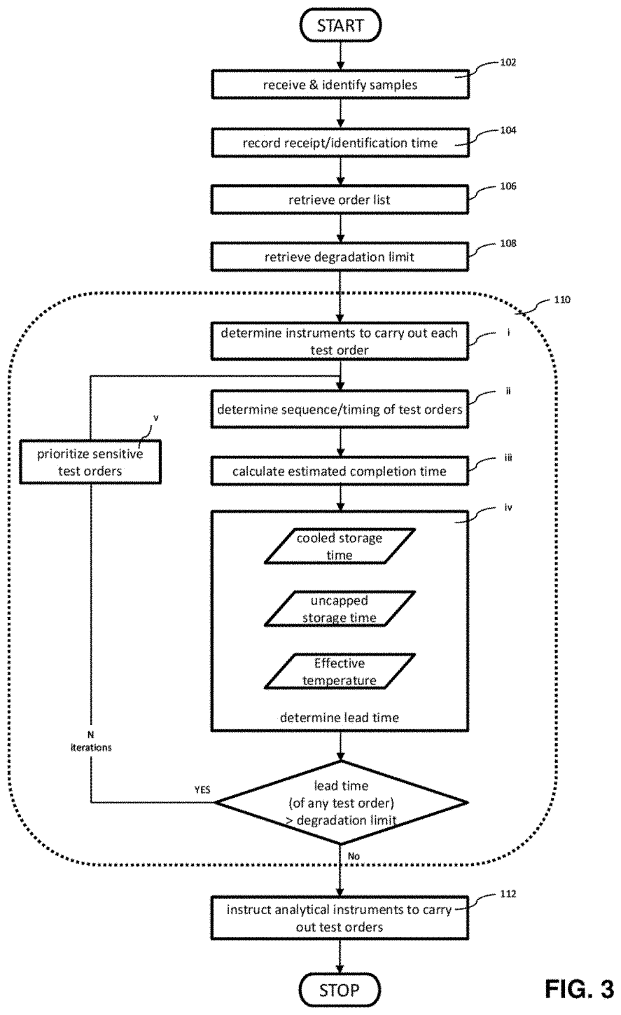

FIG. 3 is a flowchart illustrating further embodiments of the disclosed method, showing further details on the determination of the lead time corresponding to test orders. As illustrated on FIG. 3, further embodiments disclosed herein can further comprise registering a cooled storage time period during which each biological sample can be stored cooled by one or more laboratory instruments 10PRE, 10AI or 10POST comprising a refrigerated area for storing the biological sample. The registration of the cooled storage time can be based on timestamps provided by the respective laboratory instrument 10PRE, 10AI or 10POST, timestamps at which the respective biological sample was loaded respectively unloaded from the refrigerated area. Furthermore, the respective laboratory instrument 10PRE, 10AI or 10POST may provide data indicative of the actual temperature within the refrigerated area. Alternatively, or additionally, the respective laboratory instrument 10PRE, 10AI or 10POST may provide data confirming that the refrigerated area complies with cooled storage requirements (set by either a lab operator, a regulatory body or manufacture of analytical tests).

The method can further comprise, in determining the lead time corresponding to the biological samples, accounting for the cooled storage time period multiplied by a cooled degradation factor. Since biological samples can degrade at a different rate when cooled, the time spent by the biological sample(s) in a refrigerated area can be multiplied by a factor representative of the different rate of degradation when cooled. Most biological samples degrade at a slower rate when cooled; therefore, the cooled degradation factor can be less than one for such samples.

As illustrated on FIG. 3, further embodiments disclosed herein further can comprise registering an uncapped storage time of each biological sample as a time the biological sample has been in the analytical laboratory 1 uncapped. The registration of the uncapped storage time can be based on timestamps provided by the respective laboratory instrument 10PRE, 10AI or 10POST, timestamps at which the respective biological sample was uncapped respectively recapped.

The method can further comprise, in determining the lead time corresponding to the biological samples, accounting for the uncapped storage time multiplied by an uncapped degradation factor. Since biological samples degrade at a different rate when the cap is removed (exposure to ambient air) from the sample container holding the biological sample, the time spent by the biological sample(s) uncapped can be multiplied by a factor representative of the different rate of degradation when uncapped. Most biological samples degrade at a faster rate when uncapped, therefore the uncapped degradation factor can be greater than one for such samples.

As illustrated on FIG. 3, further embodiments disclosed herein can further comprise the control unit 20 receiving data indicative of an effective temperature of storage of the biological samples from one or more of the laboratory instruments 10PRE, 10AI, 10POST. Alternatively, or additionally, the control unit 20 can receive data from one or more temperature sensors located within the analytical laboratory 1 indicative of an effective temperature of storage of the biological samples such as, for example, ambient temperature. Alternatively, or additionally, the control unit 20 can receive data from temperature sensitive label(s) (passive or active e.g., RFID) attached to sample containers holding the biological samples, the temperature sensitive label(s) recording data indicative of average, maximum and/or minimum storage temperature of the biological sample. Correspondingly, the lead time corresponding to the biological samples can be determined as a function of the corresponding effective temperature. For example, the function can be an integral of the temperature curve corresponding to the biological sample. As a further example, the function may return a maximum value of a critical storage temperature has been exceeded.

According to further embodiments disclosed herein, the lead time corresponding to the biological samples can be determined as a function of the corresponding cooled storage time; uncapped storage time; and/or effective temperature, such as a weighted average function. For example,

$$storage time = 1 \times storage time at room temperature DE \text{-} CAPPED + 0.5 \times storage time at room temperature CAPPED + 0.1 \times storage time in fridge + 4 \times storage time above room temperature DE \text{-} CAPPED + 2 \times storage time above room temperature CAPPED$$

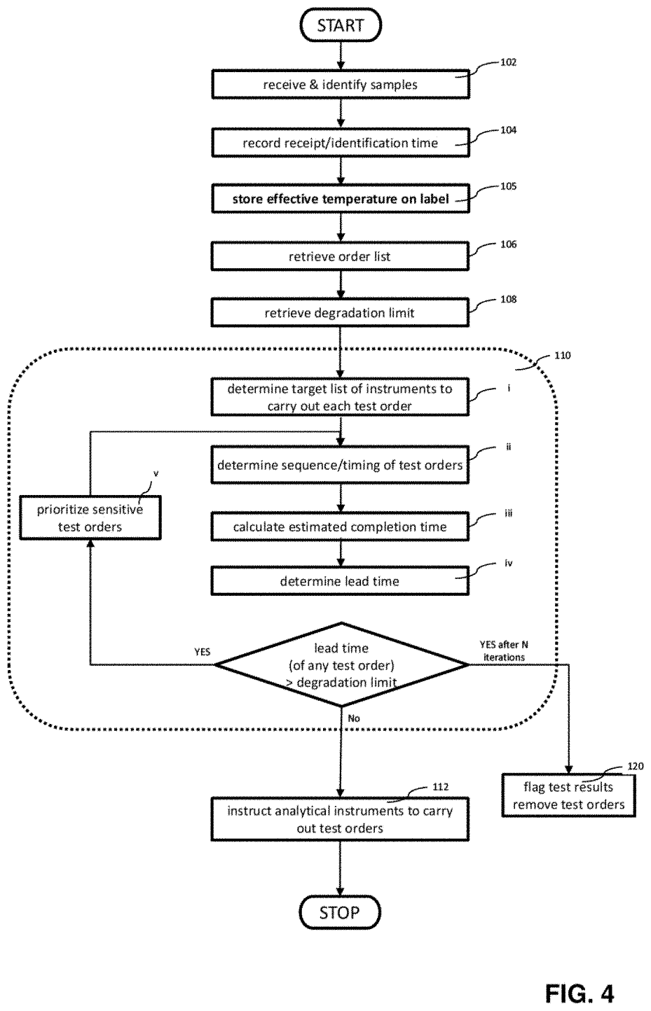

FIG. 4 shows a flowchart illustrating further embodiments of the disclosed method, wherein, in a step 120, test orders with a lead time exceeding the degradation limit can be flagged by the control unit 20. The flag can comprise data indicative of the lead time. The flagged results may then be reviewed by an expert and/or discarded. Alternatively, or additionally, the control unit 20 can remove test orders from the sample workflow with a lead time that exceeds the degradation limit. According to particular embodiments disclosed herein, the control unit 20 can be configured for receiving a manual override approving one or more sample workflow(s) despite the corresponding lead time(s) exceeding the degradation limit(s) for one or more of the test orders. In such a way, an operator can have the option to carry out the test orders despite the degradation limit(s) exceeded.

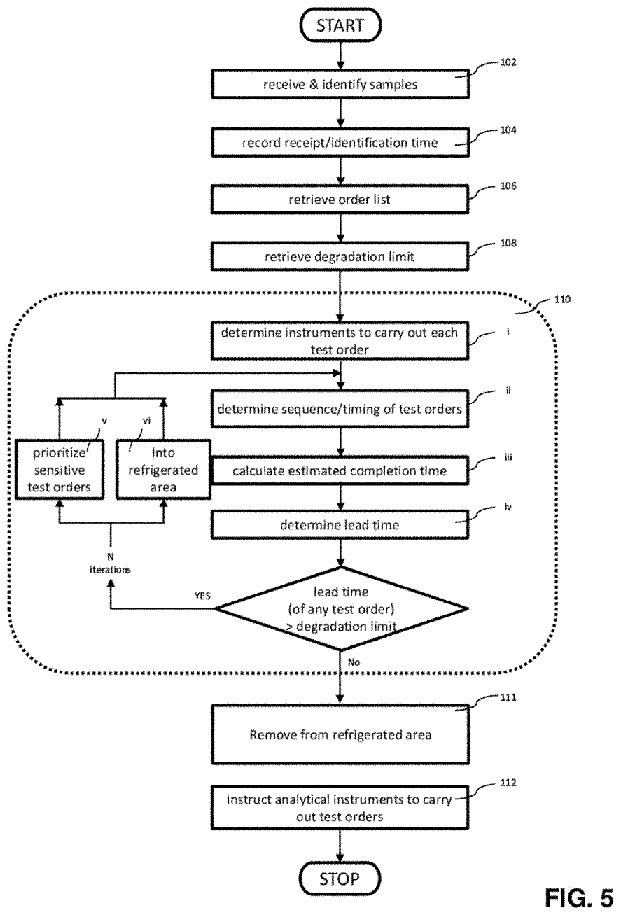

FIG. 5 shows a flowchart illustrating further embodiments of the disclosed method for an analytical laboratory 1 further comprising a sample transportation system 50 configured to transport biological samples between laboratory instrument 10PRE, 10AI or 10POST. As shown on FIG. 5, in a substep 110 vi), the control unit 20 can instruct a sample transportation system 50 of the analytical laboratory 1 to transport the biological sample into a laboratory instruments 10PRE, 10AI or 10POST comprising a refrigerated area, if the lead time exceeds the degradation limit for any of the test orders. Thereafter, having recalculated the lead time (based on cooled storage of the biological sample up until processing), in a step 111, the control unit 20 can instruct the sample transportation system 50 to transport the biological sample to the laboratory instrument 10AI, 10PRE, 10POST determined to carry out the respective test order if the lead time does not exceed the degradation limit. In such embodiments, the estimated completion time can further comprise a transportation time of the biological sample to the laboratory instrument 10AI, 10PRE, 10POST determined to carry out the respective test order.

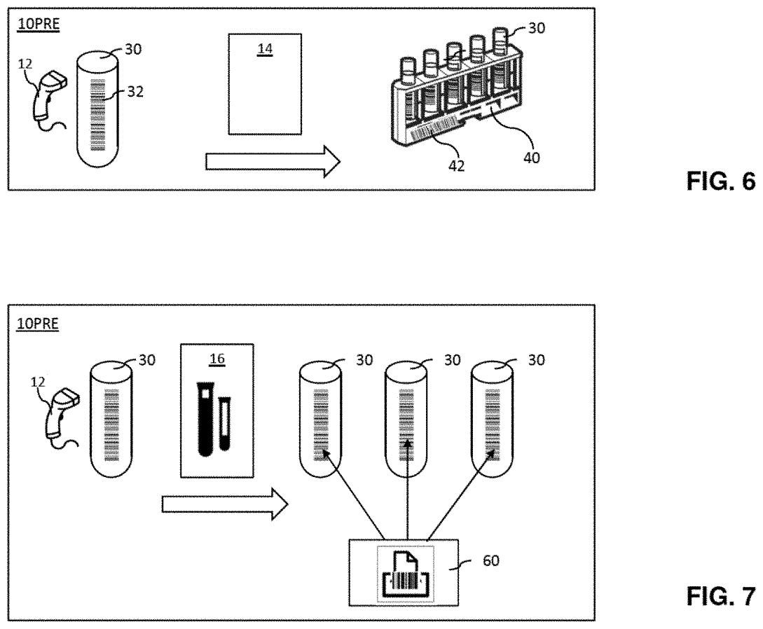

Turning now to FIGS. 6-9, particular embodiments of the laboratory instruments 10PRE, 10POST, 10AI are described.

FIG. 6 shows a pre-analytical laboratory instrument 10PRE comprising a sample container sorting unit 14 configured to sort sample containers 30 holding biological samples into sample racks 40, each sample rack 40 being identified by a rack identifier of a rack tag 42 attached to the sample rack 40, the pre-analytical laboratory instruments 10PRE being further configured to transmit signals to the laboratory control unit associating the sample identifier(s) ID of sorted sample containers 30 with the sample rack identifier(s) of the corresponding sample rack(s) 40. For embodiments where a pre-analytical laboratory instrument 10PRE sorts sample containers 30 into sample racks 40, one or more analytical laboratory instruments can be further configured to read the rack identifier Rack-ID from the rack tag 42 and transmit the rack identifier Rack-ID to the laboratory control unit with the test query.

FIG. 7 shows a further embodiment of a pre-analytical laboratory instrument 10PRE, comprising an aliquoting unit 16 configured to prepare aliquots of biological sample(s) from the sample container(s) 30 and provide each of the aliquots with a sample identifier ID on an identifier tag 32 by an identifier tag writer 60.

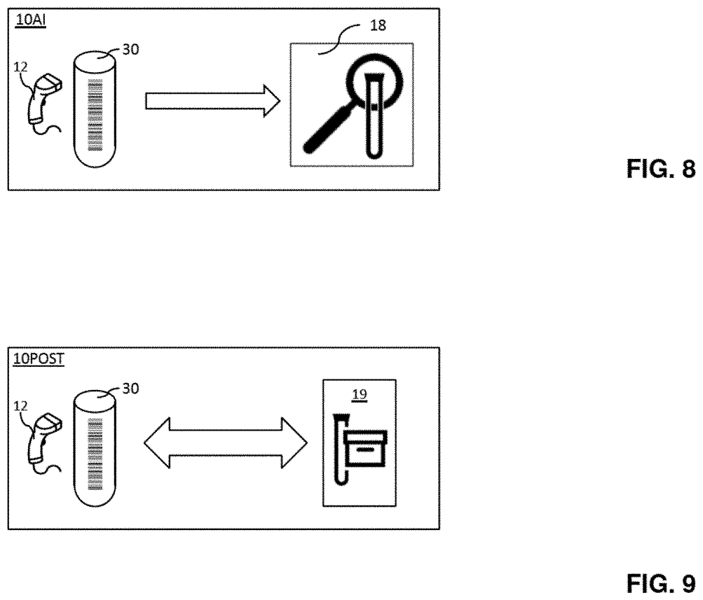

FIG. 8 shows an embodiment of an analytical laboratory instrument 10AI, comprising an analytical unit 18 configured to carry out an analytical test to measure the presence, absence and/or concentration of at least one analyte in the biological sample. The analytical laboratory instrument 10AI can perform analytical test(s) of the biological sample in response to the test order(s).

FIG. 9 shows an embodiment of a post-analytical laboratory instrument 10POST comprising a sample storage unit 19. The post-analytical laboratory instrument 10AI can be configured to store respectively retrieve sample containers 30 into respectively from the sample storage unit 19. The query by post-analytical laboratory instrument(s) 10POST to the laboratory control unit for a processing order can comprise a container to store respectively retrieve into respectively from the sample storage unit 19. Correspondingly, when queried by a post-analytical laboratory instrument 10POST, the control unit can transmit data indicative of a sample container 30 to be retrieved from the sample storage unit 19. In response to the data indicative of a sample container 30 to be stored respectively retrieved, the post-analytical laboratory instrument 10POST can store and retrieve the sample container 30 from the sample storage unit 19.

Further disclosed is a computer program product comprising instructions which, when executed by a control unit 20 of an analytical laboratory 1, can cause the analytical laboratory 1 to perform the steps of any one of the methods disclosed herein. Thus, specifically, one, more than one or even all of method steps as disclosed herein may be performed by using a computer or a computer network (such as a cloud computing service) or any suitable data processing equipment. As used herein, a computer program product can refer to the program as a tradable product. The product may generally exist in any format, such as in a downloadable file, on a computer-readable data carrier on premise or located at a remote location (cloud). The computer program product may be stored on a non-transitory computer-readable data carrier; a server computer as well as on transitory computer-readable data carrier such as a data carrier signal. Specifically, the computer program product may be distributed over a data network. Furthermore, not only the computer program product, but also the execution hardware may be located on-premise or in a remotely, such as in a cloud environment.

Further disclosed and proposed is a non-transitory computer-readable storage medium comprising instructions which, when executed by a control unit 20 of an analytical laboratory 1, can cause the analytical laboratory 1 to perform the steps of any one of the methods disclosed herein.

Further disclosed and proposed is a modulated data signal comprising instructions which, when executed by a control unit 20 of an analytical laboratory 1, can cause the analytical laboratory 1 to perform the steps of any one of the methods disclosed herein.

It is noted that terms like “preferably,” “commonly,” and “typically” are not utilized herein to limit the scope of the claimed embodiments or to imply that certain features are critical, essential, or even important to the structure or function of the claimed embodiments. Rather, these terms are merely intended to highlight alternative or additional features that may or may not be utilized in a particular embodiment of the present disclosure.

Having described the present disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

We claim:

1. A method of operating an analytical laboratory, the method comprising:
receiving and identifying a plurality of biological samples;
upon identifying the biological samples, recording data indicative of a time at which the biological samples were first identified;
retrieving by a control unit of the analytical laboratory an order list from a data storage unit comprising one or more test orders corresponding to each biological sample;
retrieving from the data storage unit by the control unit data indicative of a degradation limit corresponding to each test order of the order list;
determining by the control unit sample workflows corresponding to each biological sample and the order list; and
instructing by the control unit one or more laboratory instruments of the analytical laboratory to carry out the test orders according to the sample workflows wherein the determination of the sample workflows comprises
i) determining a target list of one or more laboratory instruments of the analytical laboratory for carrying out the one or more test orders,
ii) determining a sequence and/or timing of carrying out the one or more test orders by the target list of laboratory instruments,
iii) calculating an estimated completion time for each test order based on the sequence and/or timing of carrying out the one or more test orders,
iv) determining a lead time corresponding to each biological sample for each test order, the lead time being the time period between the time at which the biological sample was first identified and the estimated completion time of the respective test order,
v) prioritizing one or more test orders from the order list if the lead time exceeds the degradation limit corresponding to the respective test order,
repeating steps ii) to v)
until the lead time doesn't exceed the degradation limit for any of the test orders or until steps ii) to v) have been repeated for a number N of iterations.

2. The method of operating an analytical laboratory according to claim 1, further comprising,
registering a cooled storage time period during which each biological sample has been stored cooled by one or more laboratory instruments comprising a refrigerated area for storing the biological sample; and
in determining the lead time corresponding to the biological samples, accounting for the cooled storage time period multiplied by a cooled degradation factor.

3. The method of operating an analytical laboratory according to claim 1, further comprising,
registering an uncapped storage time of each biological sample as a time the biological sample has been in the analytical laboratory uncapped; and
in determining the lead time corresponding to the biological samples, accounting for the uncapped storage time multiplied by an uncapped degradation factor.

4. The method of operating an analytical laboratory according to the claim 1, further comprising,
receiving data indicative of an effective temperature of storage of the biological samples by the control unit from one or more of the laboratory instruments and/or one or more temperature sensors located within the analytical laboratory and/or a temperature sensitive label attached to sample containers holding the biological samples; and
determining the lead time corresponding to the biological samples as a function of the corresponding effective temperature.

5. The method of operating an analytical laboratory according to claim 4, further comprising,
storing by the one or more of the laboratory instruments and/or the one or more temperature sensors located within the analytical laboratory the effective temperature onto a storage label attached to the sample containers holding the biological samples; and
reading by the control unit the effective temperature from the storage labels.

6. The method of operating an analytical laboratory according to claim 1, further comprising,
flagging test results of test orders with a lead time exceeding the degradation limit by the control unit, wherein the flag comprises data indicative of the lead time; and/or
removing test orders from the sample workflow by the control unit that have a lead time exceeding the degradation limit.

7. The method of operating an analytical laboratory according to claim 6, further comprising,
receiving a manual override by the control unit approving one or more sample workflow(s) despite the corresponding lead time(s) exceeding the degradation limit(s) for one or more of the test orders.

8. The method of operating an analytical laboratory according to claim 1, wherein the of prioritization of one or more test orders from the order list comprises adjusting by the control unit the sequence and/or timing of carrying out the one or more test order(s) such that the test order with a lead time exceeding the degradation limit is carried out at an earlier time as compared to the sample workflows before the prioritization.

9. The method of operating an analytical laboratory according to claim 1, further comprising,
associating by the control unit a processing priority level with each test order;
within the prioritization of one or more test orders from the order list, increasing the processing priority level for each test order with a lead time exceeding the degradation limit; and
instructing by the control unit one or more laboratory instruments of the analytical laboratory to carry out the test orders according to the respective processing priority level.

10. The method of operating an analytical laboratory according to claim 1, wherein the data indicative of a time at which the biological samples was first identified is recorded by the control unit receiving input indicative of a time at which the biological samples were collected and one or more laboratory instrument(s) configured to receive and identify biological sample(s).

11. The method of operating an analytical laboratory according to claim 1, wherein the determination of a target list of one or more laboratory instruments of the analytical laboratory for carrying out the one or more test orders comprises determining whether the laboratory instrument is powered on and not in a low-power mode and/or determining whether all modules of the laboratory instrument required to carry out the respective test order are operational, and/or determining whether all consumables required to carry out the respective test order are available and/or determining whether all quality control and/or calibration values of the laboratory instrument are up-to-date and valid.

12. The method of operating an analytical laboratory according to claim 1, further comprising,
instructing by the control unit a sample transportation system of the analytical laboratory to transport the biological sample into a laboratory instrument comprising a refrigerated area, if the lead time exceeds the degradation limit for any of the test orders; and
instructing by the control unit the sample transportation system to transport the biological sample to the laboratory instrument determined to carry out the respective test order if the lead time does not exceed the degradation limit, wherein the estimated completion time further comprises a transportation time of the biological sample to the laboratory instrument determined to carry out the respective test order.

13. An analytical laboratory, the analytical instrument comprising:
one or more laboratory instrument(s) configured to receive and identify biological sample(s);
one or more analytical instruments configured to determine presence, absence, and/or concentration of an analyte in the biological sample; and
a control unit communicatively connected to the laboratory instrument(s), wherein the analytical laboratory is configured to carry out the method of the claim 1.

14. The analytical laboratory according to claim 13, further comprising,
a sample transportation system configured to transport biological sample(s) between the laboratory instruments, wherein the control unit is configured to carry out the method according to claim 12.

15. A computer program product comprising instructions which, when executed by a control unit of an analytical laboratory, cause the analytical laboratory to perform the method according to claim 1.
